(19) United States
(12) Patent Application Publication
(10) Pub. No.: US 2007/0014880 A1
(43) Pub. Date: Jan. 18, 2007

(54) Method for preparing ginkgo extracts having a reduced content of nonpolar plant ingredients and nonpolar impurities due to enviromental influences

(75) Inventors: Rainer Oschmann, Landau (DE); Frank Waimer, Karlsruhe (DE); Hermann Hauer, Karlsruhe (DE); Karl-Heinz Stumpf, Karlsruhe (DE)

Correspondence Address: EDWARDS & ANGELL, LLP, P.O. BOX 55874, BOSTON, MA, 02205, US

(73) Assignee: Bioplanta Arzneimittel GmbH, Ettlingen (DE)

(21) Appl. No.: 11/417,011

(22) Filed: May 2, 2006

(30) Foreign Application Priority Data
May 3, 2005 (DE) DE 102005020641.7

(51) Int. Cl. A61K 36/16

(52) U.S. Cl. 424/752

(57) ABSTRACT

The present invention relates to a method for preparing an extract from ginkgo biloba having a reduced content of nonpolar plant ingredients and nonpolar impurities due to environmental influences compared to the initial extract, the method being characterized by the following method steps:
(a) preparing an aqueous-ketonic or aqueous-alcoholic solution of an initial ginkgo biloba extract and applying the solution to an adsorber resin,
(b) eluting the adsorber resin with an aqueous C₃-C₆ ketone or an aqueous C₁-C₃ alcohol and (c) optionally concentrating and drying the extract solution thus obtained to the dry extract.

The invention further relates to an extract (liquid extract or spissum extract as well as dry extract) from ginkgo biloba having a reduced content of nonpolar plant ingredients and nonpolar impurities due to environmental influences compared to the initial extract, which is obtainable by the method of the present invention.

The present invention relates to a method for preparing an extract from ginkgo biloba having a reduced content of nonpolar plant ingredients and nonpolar impurities due to environmental influences compared to the initial (original or raw) extract, the method being characterized by the following method steps:
(a) preparing an aqueous-ketonic or aqueous-alcoholic solution of an initial extract from ginkgo biloba and applying the solution to an adsorber resin,
(b) eluting the adsorber resin using an aqueous C₃-C₆ ketone or an aqueous C₁-C₃ alcohol and
(c) optionally concentrating and drying the extract solution thus obtained to the dry extract.

The present invention further relates to an extract (liquid extract or spissum extract as well as dry extract) from ginkgo biloba having a reduced content of nonpolar plant ingredients and nonpolar impurities due to environmental influences compared to the initial extract, wherein the extract is obtainable according to the method of the present invention.

Since decades, extracts from the leaves of ginkgo biloba are used as a medicament. They are currently used for the treatment of different kinds of dementia and symptoms thereof as well as cerebral and peripheral blood circulation disorders. Ingredients, the efficacy is associated with, are terpene lactones (ginkgolides A, B, C and bilobalide) as well as glycosides of flavones (quercetin, kaempferol and isorhamnetin). However, the leaves of ginkgo biloba contain considerable amounts of components which do not contribute to the desired efficacy, but which may be responsible for risks and side effects. These are particularly nonpolar plant ingredients such as ginkgolic acids and nonpolar impurities due to environmental influences such as polycyclic aromatic hydrocarbons (PAHs). In a ginkgo extract which is efficacious and at the same time as safe as possible and as low in side effects as possible, these compounds should thus not be present to the largest possible extent.

In ginkgo leaves ginkgolic acids are contained in a concentration of about 1% based on the dry portion and dissolve to a large extent upon single extraction using aqueous ethanol or aqueous acetone such that extracts having contents of ginkgolic acids of about 5% are obtained. In addition to ginkgolic acids, further undesired nonpolar plant ingredients such as urushiols, cardanols and cardols are detectable, which inhere a risk potential as well. In the following, the entirety of these substances are designated as “nonpolar plant ingredients” and are analytically detected as ginkgolic acid as their main component on a quantity basis and their parent substance.

Ginkgolic acid elicit allergic contact dermatitis and are additionally cytotoxic, mutagenic and carcinogenic. Comparable properties are also described for cardols, whereas urushiols are extremely potent allergens (K. Schötz, Phytochem. Anal. 15, 1-8 (2004)).

Due to the existing or increasing atmospheric pollution in large parts of the world, which is a consequence of the rapidly increasing consumption of fossil fuels such as petroleum and which also concerns growing areas of ginkgo biloba, ginkgo leaves are recently provided to an increasing extent, which are polluted with considerable amounts of nonpolar impurities due to environmental influences, particularly polycyclic aromatic hydrocarbons (PAHs). In this regard, PAHs are a general term for aromatic compounds having fused ring systems such as fluorene, phenanthrene, anthracene, fluoranthene, pyrene, benz[a]anthracene, chrysene, benzo[b]fluoranthene, benzo[k]fluoranthene, benzo[a]pyrene, indeno[1,2,3-cd]pyrene, dibenzo[ah]anthracene and benzo[ghi]perylene. At least a part of the PAHs are carcinogenic and/or mutagenic such that there is a significant necessity to ensure that extracts produced from polluted ginkgo leaves are set free from these pollutants to the largest possible extent. Generally, in the case of carcinogenic substances, a lower limit, under which PAHs are considered to be harmless, cannot be defined.

Thus, it is the object of the present invention to provide a method for preparing ginkgo biloba extracts, wherein, in addition to toxicologically problematic nonpolar plant ingredients such as ginkgolic acids, also nonpolar impurities due to environmental influences such as PAHs are depleted compared to the initial extract to the largest possible extent and which further can be performed in a simple and costefficient manner. Moreover, subject of the present invention are ginkgo biloba extracts obtainable according to this method.

A ginkgo extract which has a low content of ginkgolic acids (<10 ppm and <1 ppm, respectively) is already described in EP 431 535 B1. EP 360 556 B1 describes the depletion of inactive lipophilic substances by liquid-liquid extraction, whereby an extract is said to be formed, which is inter alia essentially characterized by the absence of inactive lipophilic substances. U.S. Pat. No. 6,117,431 describes a preparation method for ginkgo biloba extracts, wherein (1.) an alcohol/water extraction is performed, (2.) the alcohol is removed, (3.) precipitated solid is separated by means of a differential centrifugation, (4.) the resulting solution is dried and (5.) lipophilic substances are separated by extracting this dry residue with supercritical CO₂. An extract containing 24% flavone glycosides, 6% terpene lactones and <5 ppm ginkgolic acids and derivatives is obtained.

A common feature of all the methods mentioned above is that there is nothing known with respect to their ability to deplete also nonpolar impurities due to environmental influences such as PAHs in addition to undesired nonpolar plant ingredients.

Also methods are known, wherein an extract is purified via adsorption on a resin and subsequent desorption (EP 692 257 B1, JP 04/182434 and EP 360 556 B1).

However, in these methods polar components are depleted by separating a polar forerun and nonpolar substances are not retained on the resin in contrast to the method according to the present invention.

In the method according to the present invention, a solution of an initial ginkgo biloba extract (initial solution) produced by an arbitrary method in a suitable solvent is applied onto an adsorber resin (step a)). The purified extract is eluted from the adsorber resin with a suitable solvent (step b)) and optionally concentrated and dried (step c)). Thereby, the nonpolar components to be removed remain on the adsorber resin and can be eluted from the resin by means of organic solvents such as acetone such that the resin can be employed again.

Preferred suitable solvents for the application to the adsorber resin (step a)) and the elution from the resin (step b)) are independently selected mixtures of water and a C₁-C₃ alcohol (methanol, ethanol, n-propanol, isopropanol) or a C₃-C₆ ketone (such as acetone or 2-butanone), particularly preferred a mixture of water and ethanol or acetone, with 30 to 70% by weight ethanol or acetone being particularly preferred.

Preferred adsorber resins are nonpolar resins on the basis of optionally substituted styrenes/divinylbenzene, such as Diaion HP-20, HP-21, Sepabeads SP-207 and SP-850. Copolymers on the basis of styrene or brominated styrene and divinylbenzene are particularly preferred.

The initial extract used to determine the initial value is obtained from the initial solution (solution of the initial ginkgo biloba extract) by drying to the dry extract.

In the method according to the present invention, the amount of the resin used as well the polarity or the composition of the solvent used have to be adjusted in such a way that the desired extract components are eluted from the resin first, whereas the undesired nonpolar plant ingredients and the nonpolar impurities due to environmental influences remain on the resin. In doing so, a higher content of water in the solvent leads to a better depletion of the undesired nonpolar plant ingredients and the nonpolar impurities due to environmental influences in the extract, i.e., they remain on the resin. Depending on the solubility of the extract, the water content of the solvent is within the above range of 30 to 70% by weight.

As initial (raw) extracts (in the form of a solution of an initial gingko biloba extract or obtained therefrom), which are to be set free from the nonpolar components via the adsorber resin, the following can be considered:
single-fold extracts prepared according to methods known per se, for example according to the European Pharmacopoeia, by extracting dried leaves of ginkgo biloba using organic solvents or mixtures thereof with water, for example using ethanol/water mixtures for acetone/water mixtures or
extracts as those described above, which are additionally already partially or largely set free from nonpolar components by means of one or more depletion steps and/or in which desired ingredients are enriched by one or more steps and which differ from the underlying single-fold extract to a greater or lesser extent (so-called special extracts).

The latter extracts can be pretreated, for example by a liquid-liquid distribution according to EP 360 556 or by some steps according to EP 431 535 such as steps (a) to (c) or by the entire method according to EP 431 535 or according to U.S. Pat. No. 6,117,431.

The solution of the initial ginkgo biloba extract according to step (a) can either be obtained directly from the preparation process of the extract or by dissolving a dry extract or another extract.

The drug-to-solvent ratio in the preparation of the initial extract (i.e., of the single-fold extract, optionally of the underlying single-fold extract in the preparation of a special extract) is generally in the range of 1:4 to 1:20, particularly in the range of 1:5 to 1:10, the extraction being carried out at a temperature in the range of room temperature (about 20° C.) to 100° C., preferably 40° C. to 60° C.

Furthermore, subject of the present invention are extracts, in particular dry extracts which are obtainable by the method according to the present invention and which are characterized by having a reduced content of nonpolar plant ingredients and nonpolar impurities due to environmental influences compared to the employed initial extracts. In the case of extracts according to the present invention, the contents of ginkgolic acids are 20 ppm at the most, preferably 10 ppm at the most and in particular 5 ppm at the most. The contents of polycyclic aromatic hydrocarbons are 50 μg/kg at the most, preferably 25 μg/kg at the most and in particular μg/kg at the most.

According to the European Pharmacopoeia, dry extracts generally have a dry residue of at least 95% by weight.

The extracts according to the present invention can be administered in the form of powders, granules, tablets, dragées (coated tablets) or capsules, preferably orally. In order to prepare tablets, the extract is mixed with suitable pharmaceutically acceptable adjuvants such as lactose, cellulose, silicon dioxide, croscarmellose and magnesium stearate and pressed into tablets which are optionally provided with a suitable coating made of, for example, hydroxymethyl cellulose, polyethylene glycol, pigments (such as titanium dioxide, iron oxide) and talcum. The extract according to the present invention can also be filled into capsules, optionally under the addition of adjuvants such as stabilizers, fillers and the like. The dosis is such that 10 to 2000 mg, preferably 50 to 1000 mg and particularly preferred 100 to 500 mg of the extract are administered per day.

Moreover, subject of the present invention are medicaments, food products and other preparations which contain these extracts, optionally in combination with other substances such as active ingredients and/or adjuvants. The term “food product” as used herein particularly refers to dietetic food products, dietary supplement products as well as medical food and dietary supplements.

### EXAMPLES

Initial solution for Comparative Example 1 and Example 1 according to the present invention

450 g of dried and ground leaves of gingko biloba with a PAH contamination due to environmental influences were extracted twice using each time seven times their weight (w/w) made up of ethanol/water 60/40 (w/w) at a temperature of 50° C.

### Comparative Example 1

### Preparation of a Single-Fold Extract as the Initial Extract

25% of the above initial solution were concentrated under reduced pressure and freeze-dried: 30.7 g (27.3% based on the dried leaves).

### Example 1 According to the Present Invention

25% of the above initial solution were concentrated and adjusted to an ethanol content of about 50% by weight. The solution (about 300 g) was filtered, applied to a column with 300 ml HP-20 resin and eluted with 900 ml 50% by weight ethanol. The eluate was concentrated under reduced pressure and freeze-dried: 25.6 g (22.8% based on the dried leaves).

Initial Solution for Comparative Example 2 and Example 2 According to the Present Invention

450 g of dried and ground leaves of gingko biloba with a PAH contamination due to environmental influences were extracted twice using each time seven times their weight (w/w) made up of acetone/water 60/40 (w/w) at a temperature of 50° C.

### Comparative Example 2

### Preparation of a Single-Fold Extract as the Initial Extract

25% of the above initial solution were concentrated under reduced pressure and freeze-dried: 30.3 g (26.9% based on the dried leaves).

### Example 2 According to the Present Invention

25% of the above initial solution were concentrated and adjusted to an acetone content of about 50% by weight. The solution (about 300 g) was filtered, applied to a column with 300 ml HP-20 resin and eluted with 900 ml 50% by weight ethanol.

The eluate was concentrated under reduced pressure and freeze-dried: 25.0 g (22.2% based on the dried leaves).

The results exhibit a significant reduction of the content of PAHs and of ginkgolic acid for Examples 1 and 2 according to the present invention compared to Comparative Examples 1 and 2.

The results are summarized in Table 1.

TABLE 1
Composition of the extracts according to the above examples

| | Extract according to | | | |
|---|---|---|---|---|
| | Comparative Example | | | |
| | 1 | | 2 | |
| | Example according to the invention | | | |
| | | 1 | | 2 |
| Fluorene [μg/kg] | 12 | 2.1 | 23 | n.d. |
| Phenanthrene [μg/kg] | 500 | 5.5 | 620 | n.d. |
| Anthracene [μg/kg] | 14 | n.d. | 20 | n.d. |
| Fluoranthene [μg/kg] | 540 | 2.9 | 640 | 3.1 |
| Pyrene [μg/kg] | 620 | 8.6 | 790 | 3.9 |
| Benzo[a]anthracene [μg/kg] | 130 | n.d. | 160 | n.d. |
| Chrysene/triphenylene [μg/kg] | 740 | 1.1 | 950 | 0.9 |
| Benzo[b]fluoranthene [μg/kg] | 150 | n.d. | 220 | n.d. |
| Benzo[k]fluoranthene [μg/kg] | 130 | n.d. | 170 | n.d. |
| Benzo[a]pyrene [μg/kg] | 17 | n.d. | 23 | n.d. |
| Indeno[1,2,3-cd]pyrene [μg/kg] | 18 | n.d. | 30 | n.d. |
| Dibenzo[ah]anthracene [μg/kg] | 2.2 | n.d. | 4.1 | n.d. |
| Benzo[ghi]perylene [μg/kg] | 11 | n.d. | 18 | n.d. |
| Total amount of PAHs [μg/kg] | 2884.2 | 20.2 | 3668.1 | 7.9 |
| Ginkgolic acids [ppm] | 29299 | 4.4 | 33148 | 17.3 |

n.d. = not detecable (detection limit: 0.5 μg/kg)

1. Method for preparing an extract from ginkgo biloba having a reduced content of nonpolar plant ingredients and nonpolar impurities due to environmental influences compared to the initial extract, characterized by the following method steps:
(a) preparing an aqueous-ketonic or aqueous-alcoholic solution of an initial ginkgo biloba extract and applying the solution to an adsorber resin,
(b) eluting the adsorber resin with an aqueous C₃-C₆ ketone or an aqueous C₁-C₃ alcohol and
(c) optionally concentrating and drying the extract solution thus obtained to the dry extract.

2. Method according to claim 1, wherein the aqueous ketone in step (a) and (b) is aqueous acetone.

3. Method according to claim 2, wherein the aqueous acetone is acetone having a content of 30 to 70% by weight.

4. Method according to claim 1, wherein the aqueous alcohol in step (a) and (b) is aqueous ethanol.

5. Method according to claim 4, wherein the aqueous ethanol is ethanol having a content of 30 to 70% by weight.

6. Method according to claim 1, wherein the water content of the solvent in step (a) and step (b) is equal or different.

7. Method according to claim 1, wherein the adsorber resin is a copolymer on the basis of styrene and divinylbenzene.

8. Method according to claim 1, wherein the adsorber resin is a copolymer on the basis of brominated styrene and divinylbenzene.

9. Extract from ginkgo biloba having a reduced content of nonpolar plant ingredients and nonpolar impurities due to environmental influences compared to the initial extract, which is obtainable by the method according to claim 1.

10. Extract according to claim 9, wherein the nonpolar plant ingredients are ginkgolic acids.

11. Extract according to claim 9, wherein the nonpolar impurities due to environmental influences are polycyclic aromatic hydrocarbons (PAHs).

12. Extract according to claim 10, wherein the content of ginkgolic acids is 20 ppm at the most.

13. Extract according to claim 10, wherein the content of ginkgolic acids is 10 ppm at the most.

14. Extract according to claim 10, wherein the content of ginkgolic acid is 5 ppm at the most.

15. Extract according to claim 11, wherein the content of polycyclic aromatic hydrocarbons is 50 μg/kg at the most.

16. Extract according to claim 11, wherein the content of polycyclic aromatic hydrocarbons is 25 μg/kg at the most.

17. Extract according to claim 11, wherein the content of polycyclic aromatic hydrocarbons is 10 μg/kg at the most.

18. Medicament, food product or other preparation characterized by a content of a ginkgo extract according to claim 9.
